(19) United States
(12) Patent Application Publication
(10) Pub. No.: US 2021/0283332 A1
(43) Pub. Date: Sep. 16, 2021

(54) INTEGRATED USER-DEFINED INPUT INTO A CLOSED-LOOP GLUCOSE CONTROL SYSTEM

(71) Applicants: Trustees of Boston University, Boston, MA (US); Beta Bionics, Inc., Concord, MA (US)

(72) Inventors: Edward R. Damiano, Acton, MA (US); Firas H. El-Khatib, Allston, MA (US); David Lim, Irvine, CA (US); Mike Rosinko, Anaheim, CA (US)

(21) Appl. No.: 17/197,420

(22) Filed: Mar. 10, 2021

Related U.S. Application Data
(60) Provisional application No. 62/987,440, filed on Mar. 10, 2020.

(51) Int. Cl. A61M 5/172; A61M 5/142; G16H 20/10

(52) U.S. Cl. CPC A61M 5/1723; A61M 5/14248; A61M 2205/502; A61M 2230/201; A61M 2205/52; G16H 20/10

(57) ABSTRACT

A controller in a system for automatic control of blood glucose level of a subject is operative to generate an insulin dose control signal for controlling insulin delivery by a delivery device, based on time-varying glucose levels of the subject as represented by an actual glucose level signal over time. The controller operates at a regular frequency to generate the insulin dose control signal to regulate the glucose levels in the subject and employs a control algorithm that functions in conjunction with and accounting for user-determined doses, such as meal doses, whose values are entered into the controller and delivered on command of a user.

### STATEMENT OF GOVERNMENT RIGHTS

The invention was made with Government Support under Contract No. DK108612 awarded by the National Institutes of Health. The Government has certain rights in the invention.

### BACKGROUND

The present invention is directed to the field of autonomous glucose level control systems. Particular background for the invention is in the following documents:

1. Damiano E R & El-Khatib F H, Fully automated control system for type 1 diabetes, WO 2006/124717 A2, Nov. 23, 2006

2. El-Khatib F H & Damiano E R, Blood glucose control system, WO 2012/058694 A2, May 3, 2012

3. Damiano E R & El-Khatib F H, Offline glucose control based on preceding periods, WO 2015/116524 A1, Aug. 6, 2016.

4. El-Khatib F H, Damiano E R, and Russell S J, Glucose control system with automatic adaptation of glucose target, WO 2017/027459 A1, Feb. 16, 2017.

The documents (1, 2) describe a control system that autonomously administers insulin doses and that allows the operator to make optional meal announcements. These meal announcements can trigger a meal dose that is determined (and potentially adjusted over time) automatically by the system. Other doses, namely bolus doses for the purpose of treating glucose excursions or basal doses for the purpose of keeping the glucose level within or close to normal range, remain autonomously determined and delivered by the control system.

### SUMMARY

As indicated above, in prior approaches the determination of all dose amounts for all dose modalities (meal, bolus, or basal doses) is handled by the system. While some users may desire such full dose-determining control by the control system, others may not draw full satisfaction from entirely giving up all dosing decisions. A common desire among the latter subgroup is to be able to quantitively determine and command their meal doses, while retaining the system's autonomy is determining and delivering bolus and basal doses.

Disclosed is a controller in a system for automatic control of blood glucose level of a subject is operative to generate an insulin dose control signal for controlling insulin delivery by a delivery device, based on time-varying glucose levels of the subject as represented by an actual glucose level signal over time. The controller operates at a regular frequency to generate the insulin dose control signal to regulate the glucose levels in the subject and employs a control algorithm that functions in conjunction with and accounting for user-determined doses, such as meal doses, whose values are entered into the controller and delivered on command of a user.

### BRIEF DESCRIPTION OF THE DRAWINGS

The foregoing and other objects, features and advantages will be apparent from the following description of particular embodiments of the invention, as illustrated in the accompanying drawings in which like reference characters refer to the same parts throughout the different views.

FIG. 1 is a block diagram of an automated glucose level control system;

FIG. 2 is a block diagram of a glucose level controller;

FIG. 3 is a schematic depiction of a sequence of user interface (UI) display pages in connection with enabling a function of user entry of meal doses for commanded delivery to a subject;

FIGS. 4-8 are graphical images of UI display pages in the sequence of FIG. 3;

FIG. 9 is a schematic depiction of a sequence of UI display pages in connection with performing the function of user entry of meal doses for commanded delivery to the subject; and

FIGS. 10-12 are graphical images of display pages in the sequence of FIG. 9.

### DETAILED DESCRIPTION

### Overview

Disclosed is an automated control system that includes an interface option by which an operator (user or care giver) can enter a dose amount that the system will attempt to deliver. The automated control system will continue to autonomously issue other (bolus and basal) insulin doses online. The system interface for entering dose amounts may enforce an upper dose bound or a set of upper dose bounds for different times of the day, which the operator could optionally set, constrained to not exceed a universal maximum dose setting in the control system. Upon the operator entering and confirming a dose amount, the control system attempts to deliver the dose and passes the delivered amount to the automated control algorithm. The control algorithm takes the dose amount into account in its subsequent autonomous dosing calculations. While the described method for entering such doses may be particularly useful for treating or compensating for food intake by the subject, the method and the control system functionality remain the same if the operator enters doses for other purposes. The described method applies equally whether the continuous glucose monitor (which provides the input glucose signal to the control system) is online or offline, e.g. the method equally applies in the system described in the previous filing (3) above. The described method could also coexist with other aspects of the system being activated or not, such as, but not limited to, having a glucose target that is adapted automatically by the system, e.g. as in the system described in a previous filing (4) above. Furthermore, the additional interface option could include features that assist the operator with performing the dose calculation, e.g. a dose calculator or wizard that allows the operator to enter their estimate of the food composition (carbohydrates, protein, fat) and that potentially leverages some personalized insulin-to-carbohydrate settings.

### Description of Embodiments

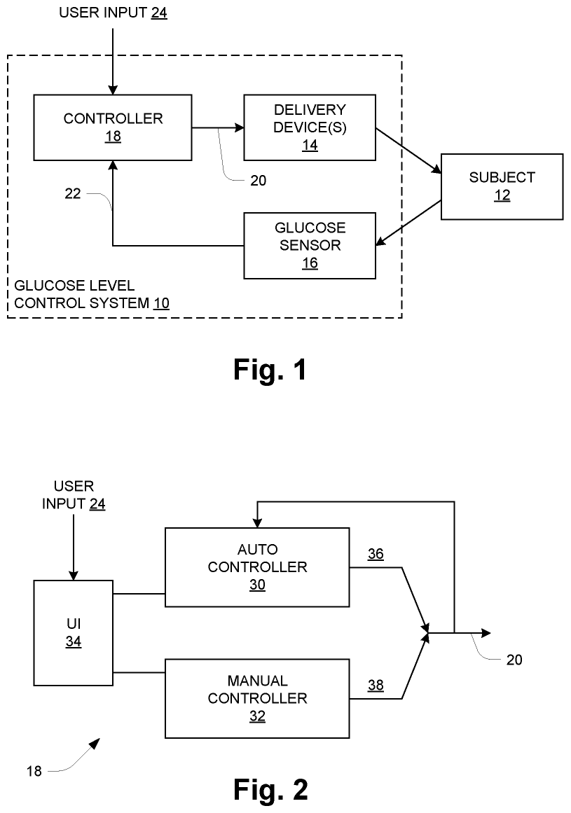

FIG. 1 illustrates an automated control system 10 for regulating the glucose level of an animal subject (subject) 12, which may be a human. The subject 12 receives doses of insulin from one or more delivery devices 14, for example infusion pump(s) coupled by catheter(s) to a subcutaneous space of the subject 12. The delivery devices 14 may also deliver a counter-regulatory agent such as glucagon for control of glucose level under certain circumstances. For the delivery of both insulin and glucagon, the delivery devices 14 are preferably mechanically driven infusion mechanisms having dual cartridges for insulin and glucagon respectively. The term “insulin” herein is to be understood as encompassing all forms of insulin-like substances including natural human or animal insulin as well as synthetic insulin in any of a variety of forms (commonly referred to as “insulin analogs”).

For autonomous operation, a glucose sensor 16 is operatively coupled to the subject 12 to continually sample a glucose level of the subject 12. Sensing may be accomplished in a variety of ways, generally involving some form of physical coupling between the subject 12 and the glucose sensor 16. A controller 18 controls operation of the delivery device(s) 14 via one or more dose control signals 20 as a function of a glucose level signal 22 from the glucose sensor 16, and subject to programmed input parameters which may be provided as user input 24 by a user such as the subject 12. One input parameter for automatic operation is the weight of the subject 12. One feature of the disclosed technique is its ability to provide effective automated control without receiving explicit information regarding either meals that the subject 12 has ingested or any other “feedforward” information, which is achieved in part by an adaptive aspect to operation of the controller 18.

For ease of description the singular term “dose control signal” 20 is used in the remainder of this description. It will be understood to encompass a collection of one or more dose control signals in the case that the system 10 employs multiple, separately controlled delivery devices 14.

The controller 18 is an electrical device with control circuitry that provides operating functionality as described herein. In one embodiment, the controller 18 may be realized as a computerized device having computer instruction processing circuitry that executes one or more computer programs each including respective sets of computer instructions. In this case the processing circuitry will generally include one or more processors along with memory and input/output circuitry coupled to the processor(s), where the memory stores computer program instructions and data and the input/output circuitry provides interface(s) to external devices such as the glucose sensor 16 and delivery device(s) 14.

The user input 24 may be provided via a local or remote user interface of some type. In one embodiment, the user interface may be provided by the controller 18 itself and resemble that of conventional insulin pumps or similar devices, e.g., by including control buttons for commanding the delivery of a bolus and perhaps a small display. In other embodiments, the system may have a wired or wireless interface to a remote device that may incorporate a fuller-function user interface, such as a smartphone or analogous personal computing device. The description herein refers to a “user” as the source of the user input 24. In one typical use, the glucose level control system 10 is a personal device worn by the subject 12 for continual glucose control. In this case the user and subject 12 are the same person. In other uses, there may be another person involved in the care of the subject 12 and providing control input, and in such a case that other person has the role of user.

Additionally, references herein to “user-determined” doses should be understood as encompassing doses determined by the user or another agent apart from the automated control algorithm of the controller 18. In some cases doses may not be completely determined by the user, such as when the user leverages a bolus wizard that may be setup for them by a health care provider for example.

In one embodiment, the controller 18 and one or more delivery devices 14 are packaged together into a single housing, which again may resemble a conventional insulin pump for example. In other embodiments these items are packaged separately and interconnected by a wired or wireless link conveying the dose control signal 20.

FIG. 2 shows the structure of the controller 18. It includes two sub-controllers shown as an automatic (AUTO) controller 30 and a manual controller 32, as well as a user interface (UI) 34. As shown, the dose control signal 20 is formed by a combination of respective dose control outputs 36, 38 of the auto controller 30 and manual controller 32.

The auto controller 30 regulates glucose level using an automated control scheme such as described in U.S. patent publication 2008/0208113A1, the contents of which are incorporated by reference herein. The manual controller 32 is used to control delivery of user-commanded doses of insulin, such as meal boluses, as described further herein. The auto controller 30 generally employs control methods or algorithms that include control parameters that are mathematically combined with reported glucose values to generate an output value that is converted (either directly or via additional conditioning) into the dose control output 36. For example, the control scheme described in U.S. patent publication 2008/02081 13A1 includes a generalized predictive control (GPC) method that incorporates a variety of control parameters. The control algorithms are generally adaptive, meaning that control parameters are dynamically adjusted during operation to reflect changing operating circumstances and a “learning” aspect—by monitoring its own operation, the algorithm adjusts its operation to be more specifically tailored to the subject 12, enhancing the algorithm's effectiveness and reducing or avoiding a need for additional explicit input information about the subject 12. In particular, the auto controller 30 tracks the delivery of insulin as reflected in values of the dose control signal 20, which includes a component corresponding to the output 38 from the manual controller 32 as well as the output 36 of the auto controller 30 itself. This tracking enables the auto controller 30 to track the accumulation of insulin in the subject 12, also referred to as “insulin on board”, to account for the effects of previously administered insulin when making current dosing decisions, as described in the above-referenced previous applications. It should be noted that certain user input 24 forms part of the control parameters used by the control algorithm; other control parameters are internal parameters according to the specifics of the algorithm, and selected ones of those internal control parameters are dynamically adjusted to realize the adaptation of the control algorithm.

In alternative embodiments the dose information of the output 38 of the manual controller 32 may be provided to the auto controller 30 in a more direct manner, i.e., by direct communication of this value from the manual controller 32 to the auto controller 30.

The remaining description focuses on certain functionality briefly mentioned above, i.e., a function by which a user can enter a dose amount that the system 10 will attempt to deliver. Such a function is particularly suited for user entry of so-called “meal boluses” at mealtimes, and thus the description employs this terminology, but it will be appreciated that the function may be used in other ways not necessarily involving meals. This function is provided by the manual controller 32 in connection with the UI 34. It will be understood that in general the auto controller 30 continues to autonomously issue other (corrective and basal) insulin doses, taking into consideration the amounts delivered by use of this user-controlled function.

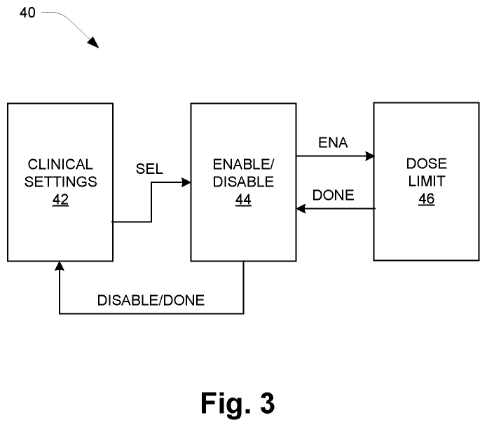

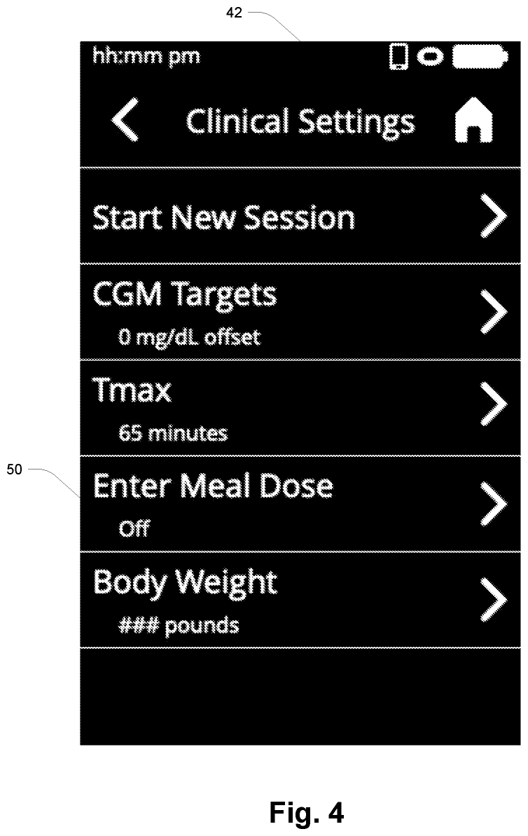

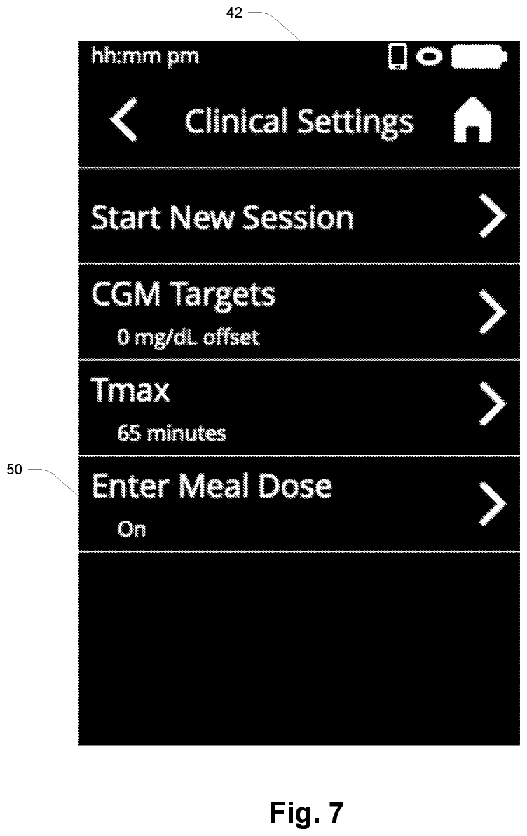

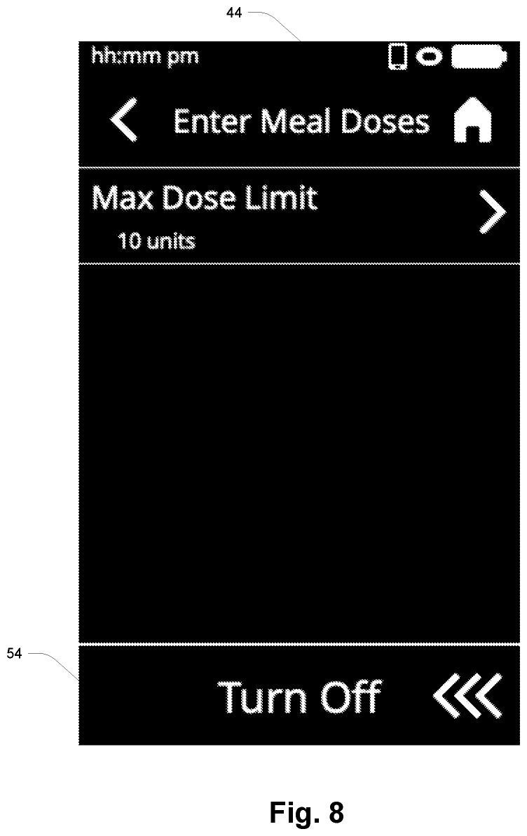

FIG. 3 shows a first sequence 40 of graphical display pages of the UI 34 used to enable the function of user entry of meal doses. These pages include a Clinical Settings page 42, an Enable/Disable page 44, and a Dose Limit page 46. Details of these pages are shown in FIGS. 4-6 (Enable flow), and FIGS. 7-8 (Disable flow).

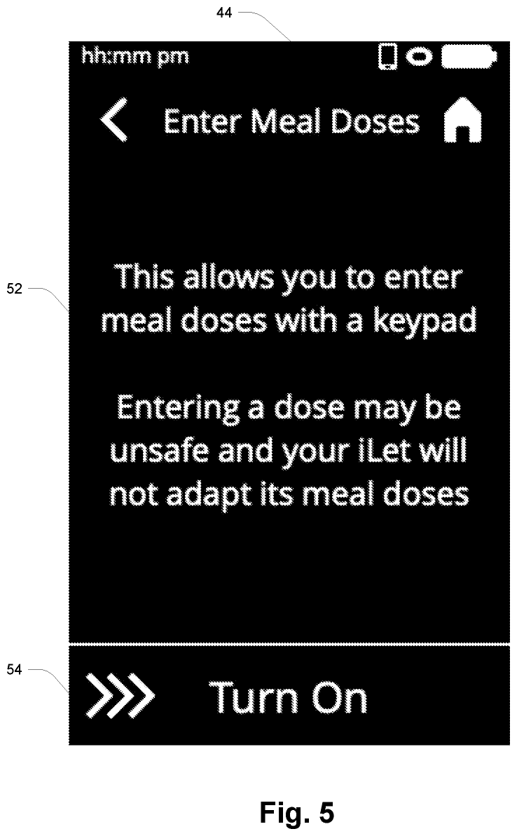

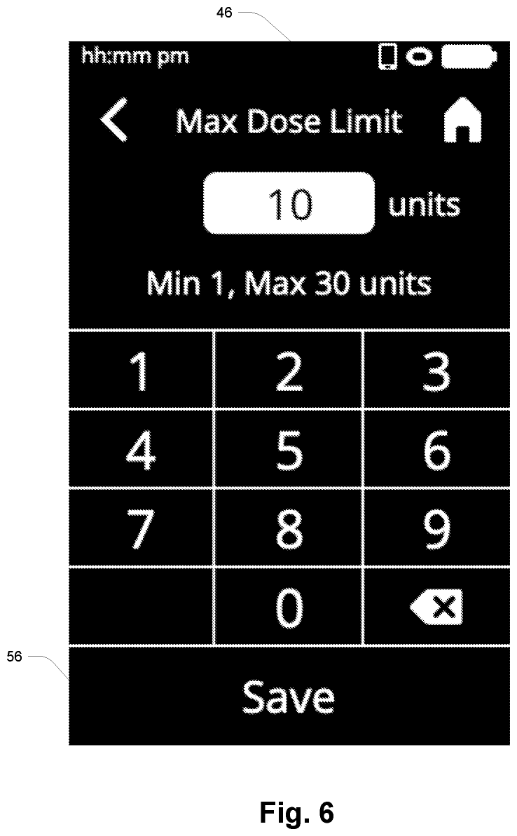

The Enable flow starts with the Clinical Settings page 42 as shown in FIG. 4, in which an Enter Meal Dose control 50 is shown as including an indication that this function is currently “Off”, i.e., disabled. Upon user selection of the control 50, the UI 34 proceeds to display the Enable/Disable page 44 (FIG. 5) which has an information area 52 and an enable/disable control 54, currently set to “turn on” (i.e., enable) the meal dose entry function. Upon user selection of the control 54, the UI 34 proceeds to display the Dose Limit page 46 (FIG. 6) which enables a user to manually enter a limit amount for this manual dose function. In this example it is assumed that the user has entered the value 10 as shown. The system may enforce more global limits on the value that it will accept, such as Min/Max of 1/30 as shown in this example. Once entry is complete, the user selects the Save control 56, and the system enables use of the meal dose function and accepts the user-entered limit for use in connection with that function, as described further below. At this point the UI 34 generally returns to a higher-level display page, such as the Clinical Settings page 42.

The Disable flow starts with the Clinical Settings page 42 as shown in FIG. 7, in which the Enter Meal Dose control 50 is shown as including an indication that this function is currently “On”, i.e., enabled. Upon user selection of the control 50, the UI 34 proceeds to display the Enable/Disable page 44 (FIG. 8) in which the enable/disable control 54 is set to “Turn Off” (i.e., disable) the meal dose entry function. Upon user selection of the control 54, the system disables use of the meal dose entry function, and the UI 34 generally returns to a higher-level display page, such as the Clinical Settings page 42.

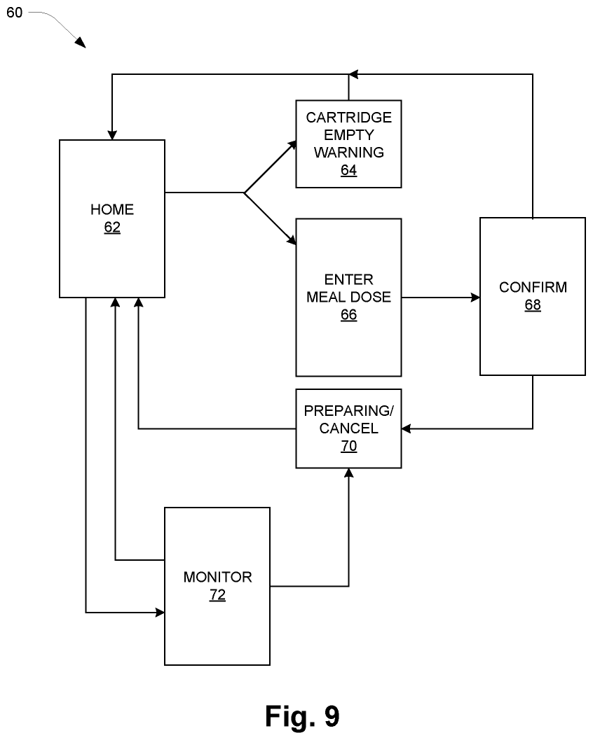

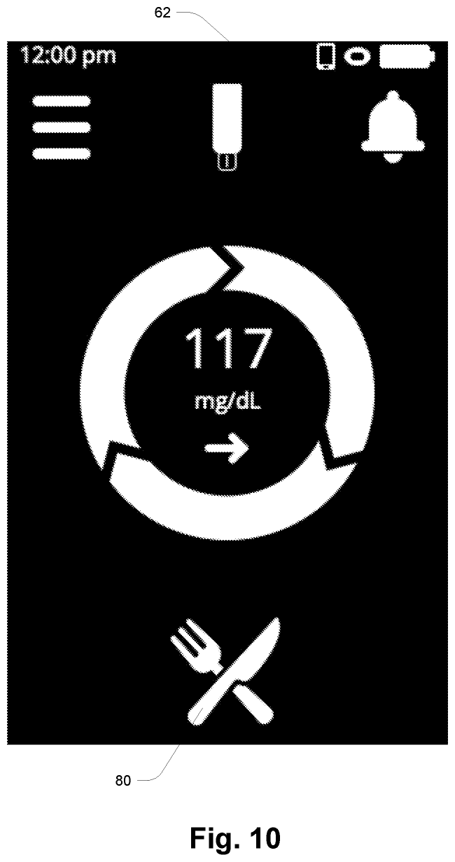

FIG. 9 shows a second sequence 60 of graphical display pages of the UI 34 used to actually perform the function of user entry of a meal dose for delivery to the subject 12. These pages include a Home page 62, a Cartridge Empty Warning page 64, an Enter Meal Dose page 66, a Confirm page 68, a Preparing/Cancel page 70, and a Monitor page 72. Details of the Home page 62, Enter Meal Dose page 66, and Confirm page 68 are shown in FIGS. 10-12.

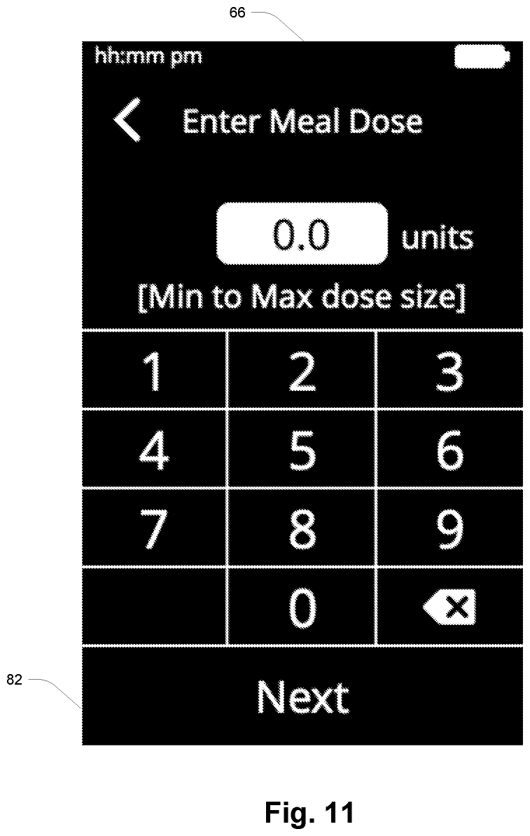

The process begins with the Home page 62 which is shown in FIG. 10. The user selects a meal dose icon 80, causing the UI 34 to proceed to the Enter Meal Dose page 66 as shown in FIG. 11. It is assumed that the meal dose function has previously been enabled in the manner described above. If not, then the meal dose icon 80 will be grayed out or otherwise unavailable, so that the function cannot be initiated. On the Enter Meal Dose page 66 (FIG. 11), the user may enter a desired value in units, which the system will accept and use subject to satisfaction of the user-entered limit during the enable process as described above.

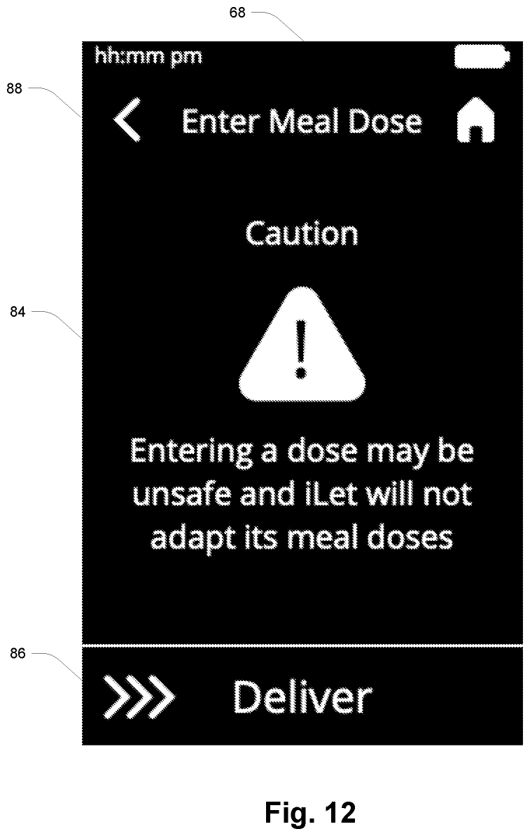

Once a satisfactory value is entered in the Enter Meal Dose page 66, the user signals completion by selecting a Next control 82, at which point the UI 34 proceeds to display the Confirm page 68 (FIG. 12). This page includes a caution/warning area 84 and a Deliver control 86. Upon user selection of the Deliver control 86, the manual controller 32 initiates delivery of the user-specified dose. Alternatively, the user may select a backup control 88 to return to the Enter Meal Dose page 66, where the user may modify the requested dose or continue to backup to effectively cancel out of the meal dose function.

Returning to FIG. 9, the process includes additional logic and UI display pages as shown and previously identified, which are outlined briefly without further detail. In the initial transition from Home 62 to Enter Meal Dose 66, the system may perform a check whether the insulin cartridge in the delivery device 14 is empty, and if so a corresponding warning page 64 is displayed and the process returns to the Home page 62 instead of proceeding to initiate delivery as described above. The process may also utilize the intermediate Preparing/Cancel” page 70 to signal to the user that the dose delivery will be beginning shortly, before the UI 34 returns to the Home page 62. The UI 34 may also provide an ability to monitor the requested delivery after it has been initiated, and utilize the separate Monitor page 72 for that purpose. The Monitor page 72 can include a live icon depicting the progress of delivery, such as by an elongated graphic component that is progressively filled or colored as delivery progresses. When the delivery is completed as requested, the UI 34 may return from the Monitor page 72 to the Home page 62. During the monitoring, there may be additional logic for (1) enabling a user to cancel the delivery in progress, and/or (2) to detect that an insulin cartridge has insufficient insulin remaining to provide the entire requested dose. In either of these cases, the UI 34 may proceed through the Preparing/Cancel page 70 to enable the user to cancel delivery, before returning to the Home page 62.

While the process of FIG. 9 assumes user entry of actual dose amounts (FIG. 11), the UI 34 may include features that assist the user in arriving at a desired dose value. As noted above, these features could include a dose calculator or wizard that allows the operator to enter their estimate of the food composition (carbohydrates, protein, fat) and that potentially leverages some personalized insulin-to-carbohydrate settings if known by the system. The calculator or wizard uses these values to calculate a corresponding dose value, which can be presented to the user for acceptance or perhaps modification before the value is used in the ensuing dose delivery process.

While various embodiments of the invention have been particularly shown and described, it will be understood by those skilled in the art that various changes in form and details may be made therein without departing from the scope of the invention as defined by the appended claims.

What is claimed is:

1. A controller for use in a system for automatic control of blood glucose level of a subject, the controller being a computerized processing device operative to generate an insulin dose control signal for controlling insulin delivery by a delivery device, the insulin dose control signal being generated based on time-varying glucose levels of the subject as represented by an actual glucose level signal over time, the controller operating at a regular frequency to generate the insulin dose control signal to regulate the glucose levels in the subject and employing a control algorithm that functions in conjunction with and accounting for user-determined doses whose values are entered into the controller and delivered on command of a user.

2. The controller of claim 1, wherein the user-determined doses are entered into the system as treatment or compensation for food intake by the subject.

3. The controller of claim 2, providing a user interface with features that assist the user in calculating the user-determined doses based on user description of the food intake by the subject.

4. The controller of claim 1, wherein the user-determined doses are limited to system-enforced dose bounds that are specified by the user, subject to system-defined outer bounds for the dose bounds.

5. The controller of claim 1, usable in both an online mode in which the actual glucose level signal is available and in an offline mode in which the actual glucose level signal is not available, wherein the user-determined doses are accounted for in both the online mode and offline mode.

6. The controller of claim 1, having two sub-controllers being an automatic controller and a manual controller having respective dose control outputs, the manual controller being operative to receive the user-determined dose values and to control delivery of the user-determined doses by generating its respective dose control output accordingly, the automatic controller operating according to the control algorithm to generate its respective dose control output accordingly, the dose control outputs of the automatic controller and manual controller being added together to generate the dose control signal for the delivery device.

7. The controller of claim 1, wherein the accounting for user-determined doses includes tracking of accumulation of insulin in the subject, and accounting for effects of previously administered insulin when making current dosing decisions.

8. The controller of claim 1, providing a user interface by which the user-determined dose values are entered into the controller and delivered on command of the user.

9. The controller of claim 8, wherein the user interface includes respective sequences by which the user first enables the delivery of user-determined doses and then enters a value for a user-determined dose and commands its delivery.

10. The controller of claim 9, wherein the delivery-enabling sequence includes a dose-limit function by which the user enters a limit value used by the controller to enforce a corresponding limit on an amount of a user-determined dose to be delivered.

11. The controller of claim 9, wherein the sequence for entering a value for a user-determined dose and commanding its delivery includes a first page for entering a dose value and a second page for accepting a user confirmation that the dose value is correct, prior to commencing delivery of the dose.

12. The controller of claim 11, wherein the first page includes a dose limit function enforcing a predetermined limit on a dose value entered by the user.

13. The controller of claim 12, wherein the predetermined limit is a limit entered by a user in a separate enable sequence used to enable delivery of user-determined doses.

14. The controller of claim 11, wherein the first page includes a dose limit function enforcing an outer bound on the user-entered limit.

15. The controller of claim 9, wherein the sequence for entering a value for a user-determined dose and commanding its delivery includes a monitoring page to enable the user to monitor delivery of a requested dose.

16. The controller of claim 15, wherein the monitoring page includes a live icon depicting progress of delivery by progressively filling or coloring as delivery progresses.

17. The controller of claim 15, wherein the monitoring page includes logic for enabling the user to cancel the delivery in progress.

18. The controller of claim 15, wherein the monitoring page includes logic for detecting that an insulin cartridge has insufficient insulin remaining to provide the entire requested dose, and cancelling the requested dose.
